(19) United States
(12) Patent Application Publication
(10) Pub. No.: US 2019/0374160 A1
(43) Pub. Date: Dec. 12, 2019

(54) HIERARCHICAL HEALTH DECISION SUPPORT SYSTEM AND METHOD

(71) Applicant: The Trustees of Princeton University, Princeton, NJ (US)

(72) Inventors: Hongxu Yin, Princeton, NJ (US); Niraj K. Jha, Princeton, NJ (US)

(73) Assignee: The Trustees of Princeton University, Princeton, NJ (US)

(21) Appl. No.: 16/475,879

(22) PCT Filed: Dec. 29, 2017
(86) PCT No.: PCT/US2017/068962

§ 371 Date: Jul. 3, 2019

Related U.S. Application Data
(60) Provisional application No. 62/442,756, filed on Jan. 5, 2017.

(51) Int. Cl. A61B 5/00; A61B 5/0205; A61B 5/01; A61B 5/0402; A61B 5/0476; A61B 5/053; A61B 5/145; A61B 5/1455

(52) U.S. Cl. CPC A61B 5/681; A61B 5/7267; A61B 5/7275; A61B 5/486; A61B 5/02055; A61B 5/01; A61B 5/024; A61B 5/0476; A61B 5/0533; A61B 5/14532; A61B 5/14551; A61B 5/4872; A61B 5/0402

(57) ABSTRACT

According to various embodiments, a hierarchical health decision support system (HDSS) configured to receive data from one or more wearable medical sensors (WMSs) is disclosed. The system includes a clinical decision support system, which includes a diagnosis engine configured to generate diagnostic suggestions based on the data received from the WMSs. The HDSS is configured with a plurality of tiers to sequentially model general healthcare from daily health monitoring, initial clinical checkup, detailed clinical examination, and postdiagnostic treatment.

### CROSS-REFERENCE TO RELATED APPLICATIONS

This application claims priority to provisional application 62/442,756, filed on Jan. 5, 2017, which is herein incorporated by reference in its entirety.

### BACKGROUND OF THE INVENTION

The present invention relates generally to health decision support systems and, more particularly, to a hierarchical health decision support system that integrates health data from wearable medical sensors into a clinical decision support system to individually track diseases based on a multi-tier structure.

Fostered by modern healthcare, human life expectancy has increased by five years in the past two decades. The healthcare system nurtures both physical and mental health of the population through clinical services and physician expertise, along with advancements in drug and prescription management. However, it also faces significant challenges. The annual admissions to registered hospitals in the U.S. have stayed above 30 million since 2014. U.S. healthcare spending reached $3 trillion in 2014, and is predicted to reach $5.4 trillion by 2024. This has spurred enormous interdisciplinary research efforts from various research communities to improve the quality of healthcare.

Despite years of remarkable progress, the healthcare system is still far from optimal. Some of the suboptimality is due to challenges like lack of adequate cancer treatments that arise from limited knowledge of the fundamental science involved. However, a substantial amount of suboptimality in the current healthcare system can be avoided, such as preventable medical errors (PMEs). A study claimed that PME is the third leading cause of death in U.S. hospitals, with more than 250,000 annual deaths, immediately after heart disease and cancer. Although the preventability of these deaths has been questioned, the consensus remains that PMEs have a severe detrimental impact on patients. Moreover, since these studies only take in-patient records into account, the actual impact of PMEs may be much worse. Therefore, effective methods need to be deployed to reduce the occurrence of PMEs.

Reducing PMEs through human inspections consumes a huge amount of effort with only weak impact. The dominant cause of PMEs is poorly-designed human-machine interfaces. More than 50% of such errors are found intimately linked to insufficient patient and drug information. Thus, computerized information systems, e.g., clinical decision support systems (CDSSs) and electronic health records (EHRs), have attracted significant research interest in the past decade. Long-term studies have shown that more than 66% of EHR-based CDSSs have significantly improved clinical practice. As a result, healthcare organizations are now adopting these systems to assist physicians and healthcare providers with intelligently filtered clinical decisions. This adoption has been further sped up by the Health Information Technology for Economic and Clinic Health (HITECH) Act of 2009, which announced a $27B federal government disbursement. Consequentially, an increasing amount of patient-specific clinical records is being collected every day, forming a fertile resource for data-oriented decision support systems.

However, conventional CDSSs are still restricted to the clinical domain. These CDSSs have very limited access to a patient's health status after the patient leaves the clinic, resulting in several deficiencies. For instance, knowledge of previous disease symptoms is an important, sometimes the only, information source for physicians and CDSSs when making decisions. However, a patient may not notice or remember all previous disease symptoms. As such, there is a need for a reliable, accurate, and intelligent out-of-clinic decision support system to complement conventional CDSSs.

Another challenge for conventional CDSSs is the non-uniformity of diagnoses offered by doctors. A typical training of a doctor in the U.S. takes more than seven years, with another three to seven years to acquire enough experience to make accurate decisions. Even though sharing experiences among doctors is possible through academic conferences and global summits, the standard deviation in doctor prescriptions can be large.

The emergence of WMSs points towards a way to address this challenge. In the past 10 years, advancements in low-power sensors and signal processing techniques have led to many disruptive WMSs. WMSs sense physiological signals passively and continuously in order to derive useful health inferences. These sensors form a powerful, yet user-transparent, human-machine interface for tracking the health condition of the user. Hence, WMSs are viewed as a promising mechanism for enabling pervasive healthcare, thus forming a suitable complement to CDSSs in a daily context.

Unfortunately, a comprehensive WMS-based information framework for health monitoring of multiple diseases is still nonexistent, greatly impeding the impact of WMSs in CDSSs. Most current WMSs utilize a simple multi-threshold method to send alerts whenever signals fall outside the specified range. However, this approach is too weak to capture enough information for the more challenging task of medical diagnosis. Diagnosis requires a stronger information extraction mechanism, such as machine learning.

Thus, there is a need for a health decision support system that bridges the information gap between data obtained from wearable medical sensors and computer-based clinical decision support systems found in clinics and hospitals.

### SUMMARY OF THE INVENTION

According to various embodiments, a hierarchical health decision support system (HDSS) configured to receive data from one or more wearable medical sensors (WMSs) is disclosed. The system includes a clinical decision support system, which includes a diagnosis engine configured to generate diagnostic suggestions based on the data received from the WMSs. The HDSS is configured with a plurality of tiers to sequentially model general healthcare from daily health monitoring, initial clinical checkup, detailed clinical examination, and post-diagnostic treatment.

According to various embodiments, a method for general healthcare utilizing a hierarchical health decision support system (HDSS) configured to receive data from one or more wearable medical sensors (WMSs) is disclosed. The method includes monitoring physiological signals to detect or track one or more diseases. The method further includes evaluating the physiological signals via an initial clinical check-up to diagnose the one or more diseases. The method also includes evaluating the physiological signals via a detailed clinical examination to further diagnose the one or more diseases. The method further includes providing post-diagnostic support based on the diagnosis of the one or more diseases.

According to various embodiments, a method for generating a disease diagnosis module is disclosed. The method includes constructing a training table from a biomedical dataset for a disease. The method further includes generating one or more decision-maker modules in a parallel fashion. The method additionally includes finalizing the disease diagnosis module.

Various other features and advantages will be made apparent from the following detailed description and the drawings.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only exemplary embodiments of the invention and are not, therefore, to be considered to be limiting its scope, the invention will be described and explained with additional specificity and detail through the use of the accompanying drawings, in which:

FIG. 1 is a table of eight supervised machine learning systems and six ensemble methods according to an embodiment of the present invention;

FIG. 2 is a schematic diagram of a hierarchical health decision support system (HDSS) according to an embodiment of the present invention;

FIG. 3 is a diagnostic decision flowchart of pervasive health decision support (PHDS) according to an embodiment of the present invention;

FIG. 4 is a table physiological signal types matched with their corresponding wearable medical sensor according to an embodiment of the present invention;

FIG. 5 is a table of the cost of sensing and storing seven common physiological signals used by PHDS according to an embodiment of the present invention;

FIG. 6 is a parallel decision flowchart of pre-laboratory clinical decision support according to an embodiment of the present invention;

FIG. 7 is an information framework for a disease diagnosis module (DDM) according to an embodiment of the present invention;

FIG. 8 is a flowchart for a DDM generation procedure according to an embodiment of the present invention;

FIG. 9 is a table of five performance parameters for a machine learning model according to an embodiment of the present invention;

FIG. 10 is a chart showing the classification accuracy for different methods according to an embodiment of the present invention;

FIG. 11 is an information framework for an arrhythmia DDM according to an embodiment of the present invention;

FIG. 12 is a table showing data instances, features, and classes in certain disease datasets according to an embodiment of the present invention;

FIG. 13 is a table showing performance result for generated DDMs according to an embodiment of the present invention;

FIG. 14 is a table showing storage requirements for diseases according to an embodiment of the present invention;

FIG. 15 is a chart showing storage changes according to an embodiment of the present invention; and

FIG. 16 is a table showing a ratio of the meta learner size and base learner size according to an embodiment of the present invention.

### DETAILED DESCRIPTION OF THE INVENTION

According to various embodiments, disclosed herein is a hierarchical health decision support system (HDSS). Its novelty lies in the combination of WMSs and CDSSs through a hierarchical multi-tier structure supported by robust machine learning tiers. The HDSS tackles both in-clinic and out-of-clinic situations in a closed-loop manner. It hierarchically and sequentially structures the information framework for daily health monitoring, automatic symptom recording, accurate clinical diagnostic support, and post-diagnostic clinical support.

The HDSS includes a recorder to store relevant raw data for symptoms to bridge the clinical information gap. With digitized memory, the problem of unreliable patient recall of symptoms can be addressed.

The HDSS further includes a scalable disease-module-based approach for monitoring diseases. Each disease is individually tracked by its disease diagnosis module (DDM). Multiple DDMs generate disease signatures in parallel to track multiple diseases simultaneously. A procedure for automatically generating DDMs from biomedical datasets may be included as well.

Different diseases (or at least disease classes) may have different signatures that can be pervasively, accurately, and efficiently obtained from the physiological signals collected by WMSs. The DDMs for all reported 69,000 human diseases would only require around 62 GB of storage in the WMS tier. This is practical for both cloud-based and base station-based WMS systems.

### Clinical Decision Support Systems and ICD-10-CM

A CDSS is an active knowledge system that stores patient-specific data and generates case-specific suggestions. It is widely used in hospitals and clinics all over the world, supported by well-developed commercial platforms, such as TheraDoc, Safety Surveillor, QC PathFinder, Sentri7, and MedMined. The primary goal of a CDSS is to help clinician adherence to suggested medical guidelines, facilitate communications with hospitalized patients, enable secure access to patient medical data, and improve the quality of general healthcare service. A CDSS is usually built alongside or directly into a local EHR, possibly employing additional input resources, e.g., picture archiving and communication systems, computerized physician order entry, e-prescribing, and positive-identification medication administration systems. Upon generating useful insights, it sends message reminders, medical orders, prompts, alerts, suggestions or dashboards to physicians.

CDSSs and EHRs rely on coding systems to track diseases electronically. To construct a coding standard, the World Health Organization introduced the International Statistical Classification of Diseases and Related Health Problems (ICD) coding system. Over the past several decades, ICD codes have successfully recorded diseases, signs and symptoms, abnormal findings, complaints, social circumstances, and external causes of injuries. Its latest version, the 10th revision (ICD-10), is a state of the art coding system used by health entities around the world. The ICD-10 used in the U.S. has two major sections: ICD-10-CM for diagnosis coding and ICD-10-PCS for in-patient procedure coding. ICD-10-CM contains approximately 69,000 disease diagnosis codes covering 20 disease categories.

### Wearable Medical Sensors

Due to rapid advancements in low-power sensing, computing, and communication, battery-powered WMSs are becoming increasingly ubiquitous. According to a report, more than 56 million wearable sensors were sold worldwide in 2015, and this number is projected to increase to 123 million by 2018. These sensors capture, store, and transmit physiological data quietly, effectively, and efficiently. The list of collectable physiological signals include, but are not limited to, heart rate (HR), body temperature (BT), respiration rate (RESP), blood pressure (BP), electroencephalogram (ECG), electrocardiogram (EEG), Galvanic skin response (GSR), oxygen saturation (SpO2), blood glucose (BG), and body mass index (BMI). This list is expanding rapidly, given the speed of ongoing technological advancements in this field.

WMS based system designs are rapidly growing. A wrist-band based sensing platform may monitor key physiological data like BP, SpO2, and ECG. Similarly, an e-textile based WMS may sense ECG, HR, and BT. A hand-held device may collect ECG and bio-impedance. Emerging smart vests incorporate inter-fabric sensor pads that may actively sense multi-lead ECG, HR, BP, and GSR with moderate data quality and decent battery life.

WMS based body-area networks (BANs) have also drawn enormous research attention. Communication protocols, transmission bandwidths, and secure encryption strategies have been discussed and analyzed for BANs. In one project, the network is carefully designed to transmit vital health signs to health providers through secure routers. Another project explores an end-to-end mobile health monitoring platform with security, communication, and quality of service guarantees.

State-of-the-art BANs enable a wide range of healthcare applications. The feasibility of using WMSs for health monitoring has been verified for hypertension monitoring, fitness tracking, and daily activity analysis. Using alert systems, these WMS based systems can make emergency calls, sending messages, emails, and/or reminders to the care providers, depending on the severity of the emergency.

### Machine Learning Systems

Machine learning systems enable computers to think and analyze problems like a human. When machine learning systems are guided by human labeling of data instances into various classes, they fall into the category of supervised learning. Supervised machine learning systems make predictions using mathematical rules learned from a labeled training dataset. Each training data instance contains a feature vector and its label as a target output. A feature represents a unique measurable phenomenon based on direct observation. To avoid feature redundancy, a feature filtering technique can be used to search, evaluate, and select only the informative features. The primary goal of a supervised machine learning system is to mathematically map feature vectors into their corresponding labels, and store this rule for future predictions. When labels represent discrete classes (continuous values) of feature vectors, the problem is referred to as classification (regression).

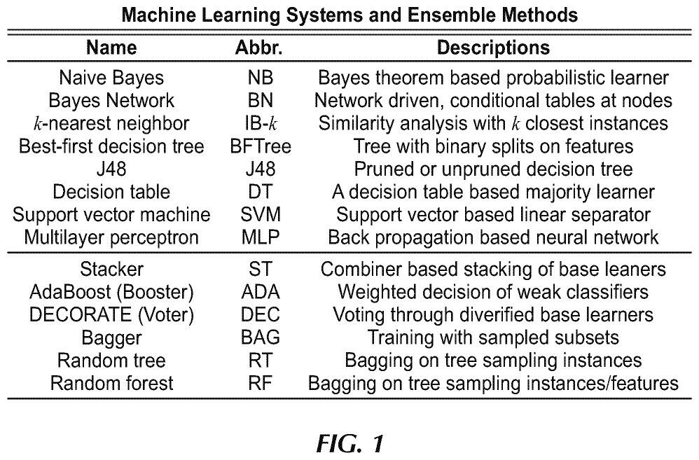

Supervised machine learning systems are useful in the healthcare domain. Disclosed herein are eight supervised machine learning systems and six ensemble methods. FIG. 1 shows their names and abbreviations, along with a short description. The upper section of FIG. 1 shows the machine learning systems disclosed herein: Naive Bayes, Bayes network, k-nearest neighbor, best-first decision tree, J48, decision table, support vector machine (SVM), and multilayer perceptron.

These supervised machine learning systems fall into three categories: similarity based, probabilistic, and error based. Similarity based machine learning systems predict the label of an incoming data instance by analyzing its similarity to pre-known data instances. For example, k-nearest neighbor predicts the class (value) of the unknown instance as the vote (mean) of most similar k instances, measured by similarity indicators such as Euclidian distance or inner product similarity in the feature space.

Probabilistic machine learning systems predict the label of an incoming instance based on probabilistic relationships between feature values and labels. For example, Naive Bayes utilizes the Bayes theorem to predict the class with the highest probability given a label-feature probabilistic relationship. As another example, Bayes network simplifies the lengthy Bayes chain calculation by introducing conditional tables. It increases the computational speed, at the expense of model size overhead. A further example, the best-first decision tree is a binary tree that splits on feature values. Selection of the best feature type to split on depends on its ability to reduce impurities, typically measured by reduction in entropy levels, termed as information gain. J48 is another example of a tree-based system in which pruning algorithms are used to avoid overfitting. Decision tables utilize rule tables to represent the probabilistic relationship between features.

Error-based machine learning systems generate mathematical models by reducing modeling errors based on various cost functions. For example, SVMs construct hyperplanes to separate data in high-dimensional feature spaces. SVMs use support vectors to maximize margins in order to reduce modeling error. As another example, a multilayer perceptron uses back propagations to train a neural network structure that minimizes an objective cost function, and predicts the label of new data instances through forward propagation.

As its name suggests, an ensemble method describes rules for a meta learner that makes a final decision based on an ensemble of decisions from base learners, such as the machine learning systems mentioned above, as nonlimiting examples. As a result, an ensemble method can significantly boost the performance of a machine learning system. The lower section of FIG. 1 shows the ensemble methods disclosed herein. A stacker stacks various types of classifiers hierarchically where high-level classifiers correct the instances incorrectly learned by low-level classifiers. AdaBoost uses a sequence of weak classifiers to generate a strong classifier, based on increasing the weights of previously misclassified data instances. DECORATE introduces diversification in the voters using artificial training examples sampled from the original dataset, but with labels inversely proportional to existing voter predictions. A new voter is added to the final voter pool only if it can pass a diversification test, where its classification results on this artificial training set differ from those of previous voters. Bagger, or a bootstrap aggregating method, trains various base learners using subsets sampled from the original training set, and makes predictions based on various rules, such as voting and averaging. Random tree samples the training instance space, and random forest samples both the training instance space and feature space. Using a bootstrap sampling rule and feature splitting method, random tree and random forest can generate ensemble structures without an external base learner. Depending on base learner types, ensemble methods can be divided into two categories: homogeneous and heterogeneous. A homogeneous method employs base learners of the same machine learning system type, whereas a heterogeneous method uses more than one machine learning system type.

### Motivation

As mentioned earlier, CDSSs are currently only employed in a clinic or hospital. Outside this domain, the system has to rely on the ability of patients to recall their symptoms, which is quite error-prone. Second, not all disease symptoms are trackable by humans, which mean they cannot be effectively fed to CDSSs. For example, symptoms of arrhythmia or diabetes are not easily noticeable by an individual. They often become evident only when something severe occurs, e.g, abrupt poundings of the chest or consistent loss of weight. Thus, it is difficult to respond in a timely and effective manner, resulting in potentially irreversible detrimental effects. These shortcomings of existing CDSSs suggest complementing them with WMS based disease diagnosis.

### Closed-Loop Hierarchical HDSS Structure

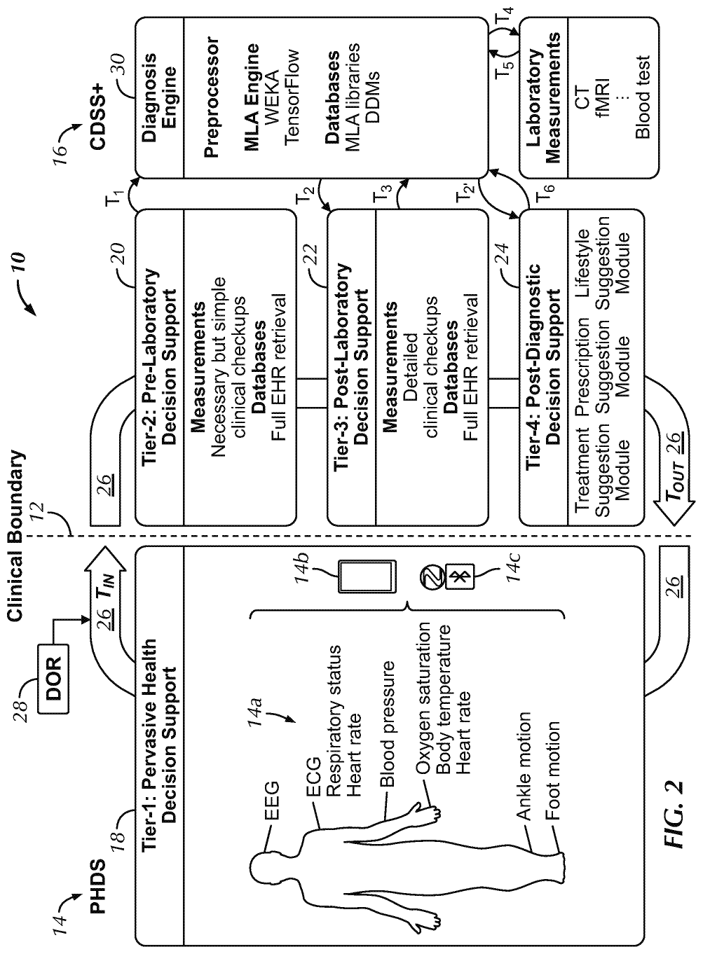

The schematic diagram of HDSS 10 is shown in FIG. 2 according to an embodiment of the present invention. HDSS 10 has two major parts, separated by a clinical boundary 12. The first part, shown on the left, is the pervasive health decision support (PHDS) module 14. The other part is PHDS-assisted CDSS module 16, denoted as CDSS+. The PHDS module 14 acts on data collected by WMSs for daily health monitoring, while CDSS+ 16 assists with clinical decisions.

The PHDS module 14 includes one or more WMSs 14a, a device 14b for collecting data, and a network system 14c for connecting the WMSs 14a to the device 14b. As mentioned earlier, the WMSs 14a include sensors configured to collect physiological signals including, but not limited to, heart rate (HR), body temperature (BT), respiration rate (RESP), blood pressure (BP), electroencephalogram (ECG), electrocardiogram (EEG), Galvanic skin response (GSR), oxygen saturation (SpO2), blood glucose (BG), and body mass index (BMI). The device 14b may be implemented in a variety of configurations including general computing devices such as desktop computers, laptop computers, tablets, networks appliances, or mobile devices such as mobile phones, smart phones, or smart watches, as nonlimiting examples. The device 14b includes one or more processors for performing specific functions and memory for storing those functions. The network system 14c may be implemented as a single network or a combination of multiple networks. Network system 14c may include but is not limited to wireless telecommunications networks, WiFi, Bluetooth, Zigbee, or other communication networks.

HDSS 10 has four major tiers, as shown in FIG. 2. It uses these tiers to sequentially model general healthcare from daily health monitoring, initial clinical checkup, detailed clinical examination, and post-diagnostic treatment.

Tier-1 18 assists with daily health monitoring. Tier-1 18 incorporates decision modules trained using clinical domain knowledge, and transmits information across the clinical boundary 12. This helps individuals, even those without professional medical training, track their diseases.

Tier-2 20 provides immediate decision support to physicians for an incoming patient. At Tier-2 20, physician insight and basic measurements provide additional inputs, even though very accurate laboratory results are not yet available.

At Tier-3 22, a more detailed diagnostic analysis becomes feasible based on laboratory measurements.

Finally, Tier-4 24 delivers post-diagnostic treatment, prescription, and lifestyle suggestions.

HDSS 10 operates from Tier-1 to Tier-4 in a sequential and closed-loop manner, as indicated by the large arrow 26 connecting all four tiers in FIG. 2. Hence, operation and information flows in the loop are directional. Tier-x data are available to Tier-y, where x<y, however, the reverse is not true. Thus, subsequent tiers gather more information than previous tiers, but typically with higher time and energy costs.

To make sure HDSS 10 can smoothly transfer information across tiers, the tiers are interconnected via transitions (T), depicted by indexed arrows T_{IN}, T_{OUT}, and T₁ through T₆ in FIG. 2. When an alert is raised at Tier-1 18, a transition T_{IN} crosses the clinical boundary 12 and transfers patient information to Tier-2 20. Along with T_{IN}, Tier-1 18 passes relevant symptom records stored as disease-onset records (DORs) 28 for subsequent analysis.

At Tier-2 20, the data, aggregated with additional measurements and physician insights, are passed to the diagnosis engine 30 through T₁. In one embodiment of HDSS 10, decision-making processes are allocated to an external diagnosis engine 30, so that modifications to existing CDSSs can be minimized. However, alternative embodiments of HDSS 10 may have a diagnosis engine 30 integrated in the system. The diagnosis engine 30 includes one or more processors for performing specific functions and memory for storing those functions, as illustrated by machine learning system libraries and DDMs in databases accessible by machine learning system engines, such as WEKA and TensorFlow as nonlimiting examples. The diagnosis engine 30 may be implemented in a variety of configurations including general computing devices such as desktop computers, laptop computers, tablets, networks appliances, or mobile devices such as mobile phones, smart phones, or smart watches, as nonlimiting examples. The diagnosis engine 30 may also be integrated into device 14b or may be a separate device.

There are two possible outgoing transitions from the diagnosis engine: T₂ and T_{2′}. When further laboratory measurements are needed to make a final diagnosis, T₂ transfers HDSS 10 to Tier-3. Otherwise, HDSS 10 reaches Tier-4 24 through T_{2′}. Regardless of which transition is selected, diagnostic suggestions from the diagnosis engine 30 and information on whether further laboratory measurements are needed are immediately available to physicians at Tier-2 20.

When T₂ occurs, Tier-3 22 calls the diagnosis engine 30 through T₃. Appropriate laboratory tests are ordered through T₄, and reports fed back to the diagnosis engine 30 through T₅. At this stage, a more detailed disease-specific diagnosis can be performed. Diagnostic analysis at Tier-3 22 is more time-consuming and expensive than at Tier-2 20. Laboratory measurements can be slow and expensive. For example, a blood test report can take as much as 12-16 hours, and reports for computed tomography (CT) and functional magnetic resonance imaging (fMRI) tests are even slower. However, Tier-3 22 is still the most important tier since it has all available information for making a concrete diagnosis.

Tier-4 24 is reached through T_{2′} or T₆. All data measurements, extracted features, and diagnostic suggestions from previous tiers are passed on to Tier-4 24 for making post-diagnostic suggestions. Upon a satisfactory outcome, a final transition, T_{OUT,} indicates completion of the clinical visit and transfers the HDSS 10 state back to Tier-1 18 PHDS 14.

### Tier-1 18—Pervasive Health Decision Support (PHDS) 14

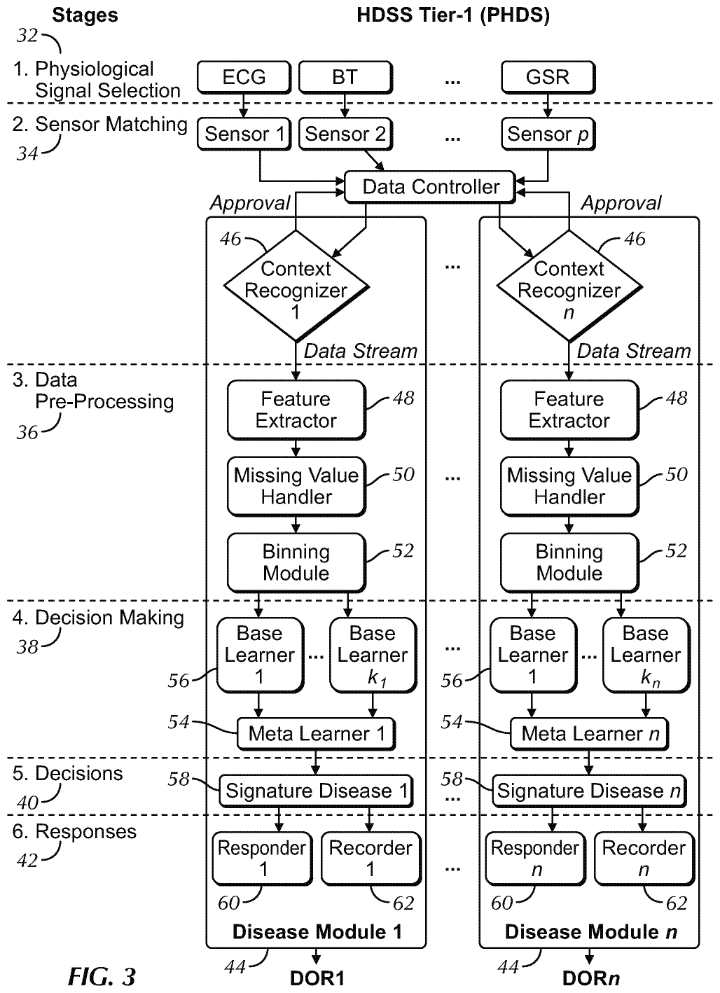

This tier uses WMS data to detect/track multiple diseases. As shown in FIG. 3 the diagnostic decision flow of PHDS 14 is shown using six sequential stages: (1) selection of target physiological signals 32, (2) matching of these signals with their WMSs 34, (3) pre-processing of the collected signals for machine learning models (MLMs) pre-trained using machine learning systems 36, (4) decision making through MLMs 38, (5) obtaining disease signatures 40, and (6) responding according to the decisions 42. Diagnosis of disease i is done through its own tier-wise disease module 44. Using this structure, PHDS 14 can monitor any number n diseases in parallel.

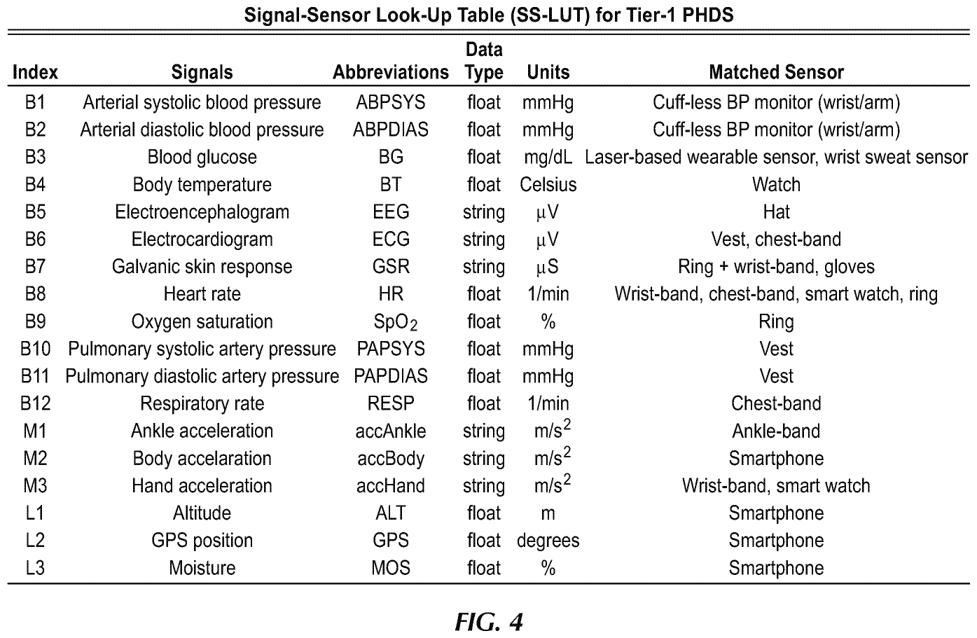

In the first stage 32 and the second stage 34, the p physiological signal types are matched with their corresponding WMSs through a signal-sensor look-up table (SS-LUT), as shown in FIG. 4. The signals are divided into B-series (physiological), M-series (motion), and L-series (location). A context recognizer 46, shown at the top of each disease module 44, uses the M-series and L-series data to validate the context for B-series data.

For example, muscle movement generates noise in ECG measurements. Consequentially, a clinical 12-lead ECG measurement requires the patient to stay still for at least 30 seconds. In Tier-1, however, such a constraint on the users is impractical. WMS data collected during fitness training may be too noisy for arrhythmia analysis, and thus may need to be flagged as such.

In the third stage 36, the digitized signals from WMSs are pre-processed from possibly incomplete and inconsistent raw measurements into formats understandable by MLMs. Upon approval from the context recognizer 46, the appropriate data streams are fed to a disease module 44 based on the SS-LUT indices specified by the module 44. A feature extractor 48 computes the desired features from these data streams. Then, a missing value handler 50 gets rid of missing feature values using statistical methods, such as value imputations and interpolations. Finally, a binning module 52 maps continuous measurements to separate bins, thus reducing overall computational load on the MLMs.

Diagnostic decisions are made in the fourth stage 38. In PHDS 14, diagnosis of disease i is done using its pre-trained MLM that includes a meta learner 54 and kᵢ base learners 56. The meta learner 54 finalizes a prediction based on outputs from kᵢ base learners 56, using the ensemble method selected during the training phase. Note that sometimes the training phase may prefer a single base learner 56, thus obviating the need for a meta learner 54, given the fact that ensemble methods do not always boost MLM performance.

The fifth stage 40 obtains the binary signatures 58 of the n diseases being targeted. A signature of 0 indicates benign status, and a signature of 1 indicates potential disease onset.

A series of 1s triggers a responder 60 and a recorder 62 of the disease in the sixth stage 42. The recorder 62 stores the raw measurements that led to this disease diagnosis in a DOR for future clinical usage. The responder 60 assists the users with appropriate medical suggestions for this disease, and directs them to the most relevant part of the clinic. This transition from PHDS 14 to clinic saves an enormous amount of time for both physicians and patients by completely bypassing the lengthy initial status-checking procedure performed in the clinic.

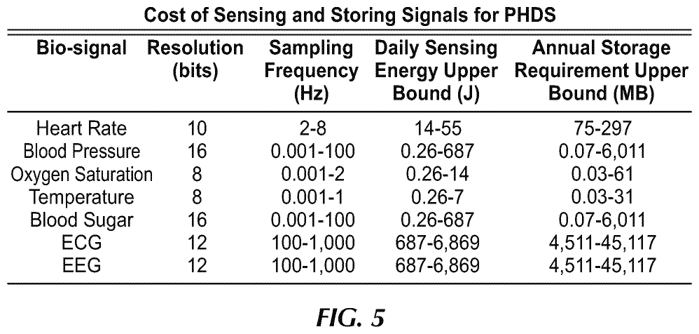

FIG. 5 presents the cost of sensing and storing seven common physiological signals used by the PHDS 14. These values were obtained from WMSs based long-term continuous health monitoring schemes. As shown, both the energy and storage costs are modest and well within the capabilities of current technologies. Note that signature-driven DORs only need to store raw measurements that trigger disease diagnosis. Since this can be expected to be quite infrequent, these values in fact only indicate a cost upper bound.

### Tier-2 20—Pre-laboratory Clinical Decision Support

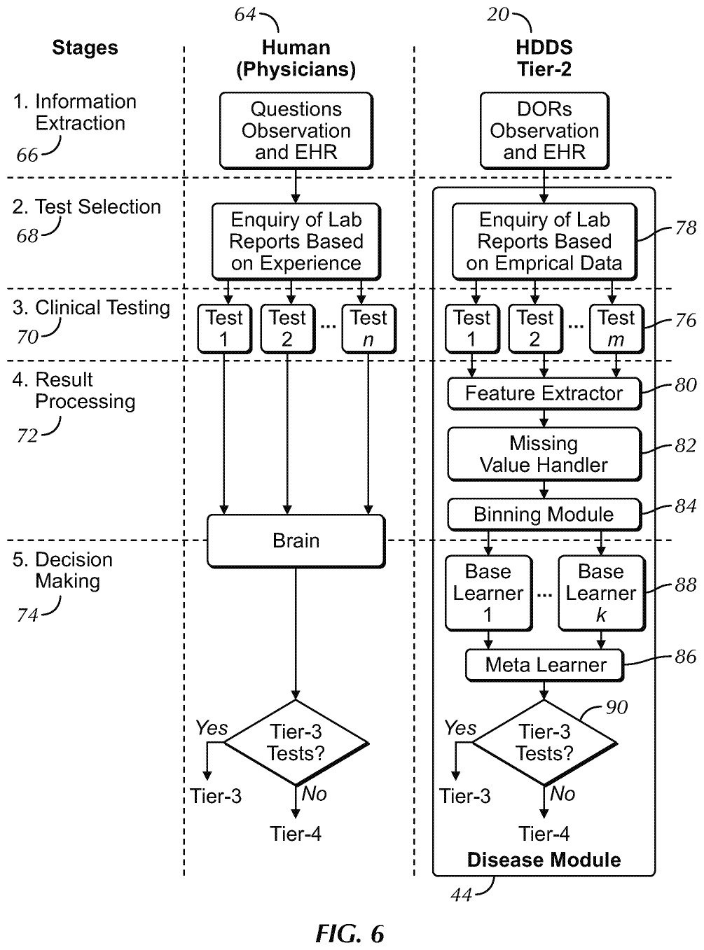

Tier-2 20 helps physicians evaluate incoming patients in a clinical setup. FIG. 6 shows the parallel decision flows of Tier-2 20 and a physician 64. The decision flows have five stages: (1) information extraction 66, (2) test selection 68, (3) clinical testing 70, (4) result processing 72, and (5) decision making 74.

During a patient's first clinical checkup, physicians extract background information on the patient through a series of questions, observations, and a review of EHR. The effectiveness of this information extraction process depends heavily on physician experience. Physicians may also utilize n Tier-2 tests and measurements to acquire more relevant disease indicators. The physician processes the aggregated information in his/her brain, and decides whether further Tier-3 tests are needed or if a diagnosis can be finalized. Due to different physician-specific styles of practice and inherent subjectivity, this decision making process causes inter-physician practice variations.

Tier-2 20 has a similar decision flow. Disease diagnosis is carried out using the disease module 44 shown in FIG. 6. As opposed to the conventional information extraction process used by physicians, Tier-2 20 utilizes EHRs and DORs to inform the physicians of a patient's background information and a detailed symptom record. The DOR symptom types can be updated based on the latest domain knowledge to provide a uniform picture to all physicians. This aspect cannot be accomplished simply by a physician. Tier-2 20 recommends m tests 76 based on the collected empirical data 78. Since a physician may not remember all available m tests at a given time, Tier-2 HDSS includes m≥n. As in the case of Tier-1 18, the test measurements are pre-processed (via feature extractor 80, missing value handler 82, and binning module 84) before being fed to a meta learner 86 through k base learners 88. Finally, Tier-2 20 transfers control to either Tier-3 22 or Tier-4 24 depending on whether further laboratory measurements are necessary, as shown by inquiry 90

### Tier-3 22—Post-laboratory Clinical Decision Support

In Tier-3 22, the main objective is to make an accurate diagnosis with help from all available medical measurements. Compared to previous tiers, diagnosis in this tier can be narrowed down to within a few similar disease sub-types. In other words, where Tier-1 18 and Tier-2 20 perform horizontal monitoring of many diseases, Tier-3 22 performs a deep vertical analysis into a specific disease candidate.

Tier-3 22 uses the same decision flow as Tier-2 20; however, the information extraction stage is removed and a modified clinical testing stage included. The tests are now expanded from the immediate and simple clinical tests to all relevant complex and sophisticated, but more informative, laboratory tests. These tests may require extra assistance, processing time, equipment, and experiments, such as blood tests, CT, and fMRI. Though quite beneficial, these tests incur significantly higher time and expense. Therefore, the exact allocation of tests to these tiers needs to be updated periodically and, in fact, be decided based on facilities available in the clinic.

### Tier-4 24—Post-diagnostic Decision Support

Tier-4 24 provides post-diagnostic decision support, where treatments, prescriptions and medications, and future lifestyle suggestions, assisted via machine learning systems, can be generated by the respective modules. In this Tier-4 24, it is much harder to derive a unified decision flow or information framework, as these modules have different final objectives and serve various end-users. However, all these modules share the same need for diagnostic data from previous tiers.

Based on information from Tier-2 20 or Tier-3 22, if the health status of the patient is satisfactorily resolved, T_{OUT} transfers control across the clinical boundary 12. CDSS+ 16 finalizes the clinical visit by appropriately updating EHRs, generating patient-centered lifestyle recommendations, such as saturated fat and cholesterol intake restrictions, and transferring HDSS 10 back to the PHDS 14 tier to initialize a new monitoring round.

### Disease Diagnosis Modules

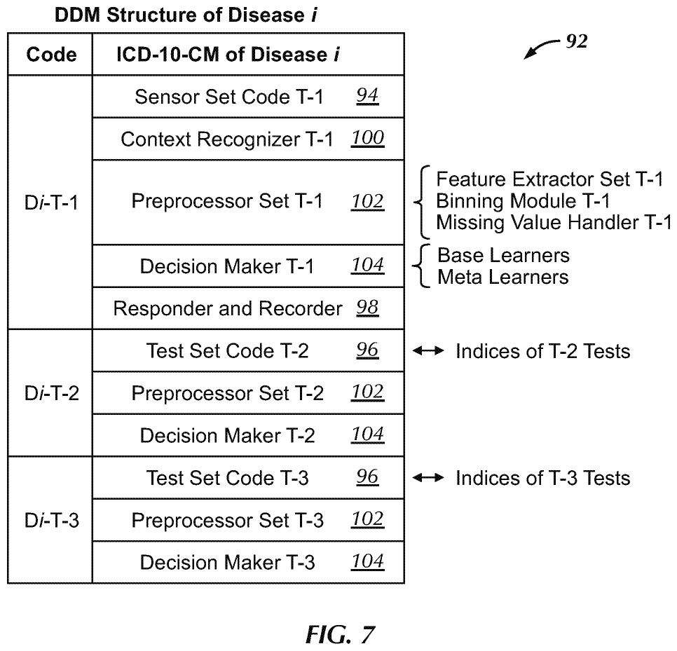

As mentioned earlier, diseases can be tracked by their independent DDMs in each tier. A DDM specifies the unique and necessary information framework components used by HDSS 10 for diagnosis. Hence, to evaluate or update the diagnostic rule for a given disease, one only needs to modify its DDM instead of restructuring the entire HDSS 10. DDMs share the standardized information framework shown in FIG. 7. A DDM 92 includes a unique code and its tier-wise disease modules. In order to be consistent with existing CDSSs and EHRs, the DDM code is set to the ICD-10-CM code of the disease. The disease module for disease i at Tier-j is denoted as Di-T-j, j=1, 2, 3. The sensor set code 94 and test set codes 96 communicate with external electronic systems. The sensor set code 94 works with SS-LUT to specify the WMSs needed for diagnosis of disease i. The test set code 96 searches through the clinical test directory for desired laboratory measurements. The responder and recorder component 98 corresponds to responders and recorders. The context recognizer 100, preprocessor 102, and decision makers 104 represent the intelligent MLM sectors. A decision maker 104, which stores the MLM, makes diagnostic predictions based on the latest domain knowledge extractable from an up-to-date training dataset on disease i, and thus acts as the core of a DDM. The preprocessor set 102 for a decision maker 104 stores its corresponding feature extractor, binning module, and missing value handler. Once a decision maker 104 is generated through the training process, its corresponding preprocessor set 102 can be obtained simultaneously.

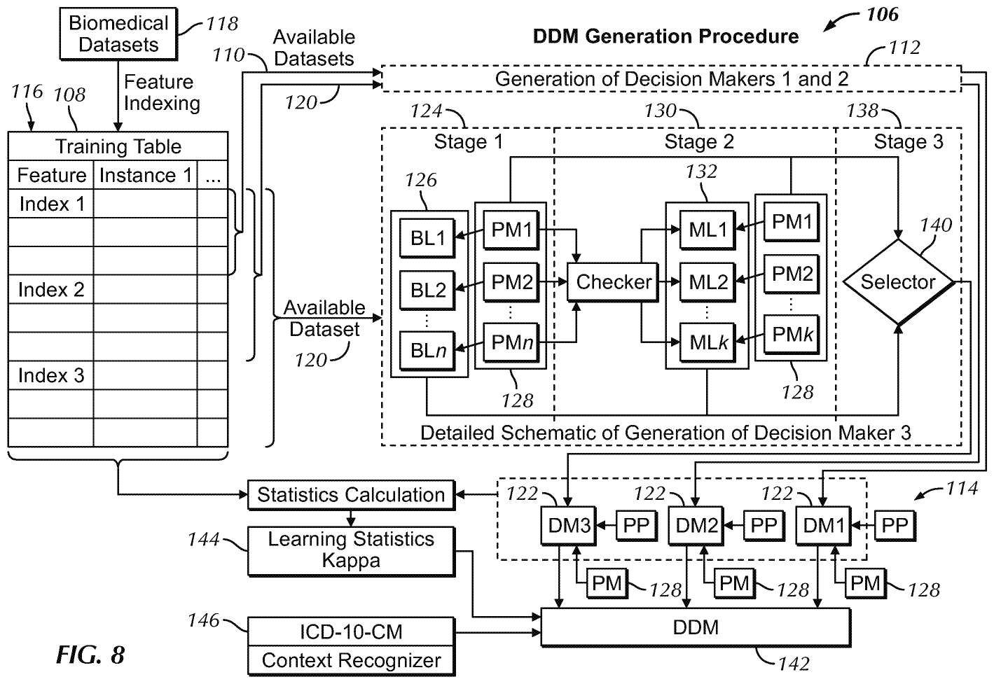

A DDM generation procedure 106 is utilized to automatically generate DDMs. As shown in FIG. 8, a training table is sequentially constructed from a biomedical dataset for disease i 108, tier-wise available datasets are derived 110, decision makers are generated in a parallel fashion 112, and the DDM for disease i is finalized 114.

A training table 116 for disease i can be acquired from a biomedical dataset 118 after the feature indexing process is complete. Each feature is given an availability index in the form of an integer ranging from 1 to 3 to signal its availability at Tier-1 18 through Tier-3 22. A decision maker (DMx) 122 at Tier-x can only be trained using features with indices t, where t≤x. As shown by the vertical parentheses in FIG. 8, data instances containing available features at a given tier form an available dataset 120 for that tier. This available dataset 120 can later be used to generate the decision maker 122. Since the training table 116 can simultaneously support multiple available datasets 120, decision makers 122 can be generated in a completely parallel manner.

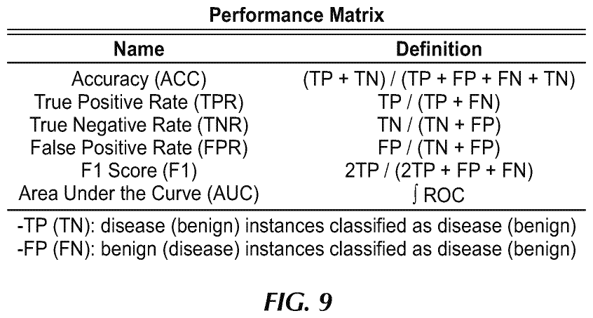

Given an available dataset 120, there are three major stages in decision maker generation 112, as shown in the middle section of FIG. 8. Stage 1 124 generates a set of base learners 126 and their performance parameters. In one embodiment, a performance matrix 128 is utilized to store five important performance parameters for a base learner 126, as shown in FIG. 9. Accuracy (ACC) indicates the base learner's overall prediction capability. The true-positive rate (TPR) and true-negative rate (TNR) measure the base learner's capability to recognize disease and benign cases, respectively. The false positive rate (FPR) specifies the percentage of false alarms generated by the base learner. The F1 score measures the overall performance of these two rates. The area under the curve (AUC) is another accuracy metric, which uses a receiver operating characteristic (ROC) curve to capture the tradeoffs between the TPR and false positive rate (FPR). A value close to 1 is preferred for ACC, TPR, TNR, F1, and AUC.

Stage 2 130 generates a series of meta learners 132 using base learners 126 passed from Stage 1 124. To provide guidance to this process, performance matrices 128 are checked by a checker 136 to match base learner candidates with appropriate ensemble methods, e.g., AdaBoost prefers weak base learners and voters prefer diversified base learners.

In Stage 3 138, a statistical selector 140 compares performance matrices 128 of all generated learners 132 from both stages 124 and 130 based on pre-defined statistical criteria, such as TPR, TNR, F1 score, McNemar metric, geometric mean error, and win/draw/loss game, and selects the best learner as the final decision maker 122.

The generated decision makers 122 are packaged into the final DDM 142, together with their performance matrices 128, and their learning statistics 144. Storing the learning statistics 144 is important to effectively defend against an adversarial machine learning attack, which aims to negatively impact the decision making process by degrading the performance of MLMs. Finally, the complete DDM 142 is stamped with its ICD-10-CM code 146, and uploaded to HDSS 10.

### Evaluating HDSS Performance According to an Embodiment of the Present Invention

This section evaluates the performance of a disclosed HDSS according to an embodiment of the present invention. First, it is shown how an arrhythmia DDM may be constructed based on its biomedical dataset. Then DDMs for five other diseases are evaluated to demonstrate the scalability of HDSS and the feasibility of using WMSs for disease diagnosis. The HDSS storage requirements for about all known 69,000 human disease are also estimated.

Arrhythmia is used as a nonlimiting example to show how the DDM generation procedure works. The arrhythmia dataset was acquired from the openly accessible UCI repository. It contains 452 data instances, each of which stores 279 feature values extracted from a 12-lead ECG recording. Instances are either labeled as benign or one of 15 arrhythmia subtypes.

A given biomedical dataset is transformed into a training table with the feature indexing process. Feature indexing at Tier-1 depends on the available WMS types in SS-LUT that can be matched to the signals in the biomedical dataset. According to one embodiment, a 12-lead smart vest is selected as the WMS for ECG collection. This WMS was picked to obtain the highest achievable accuracy in the WMS tier, although other WMSs may be selected in other embodiments. With access to all 12 ECG channels in Tier-1, Tier-2, and Tier-3, the 279 features are available in all tiers. Hence, all the features are labeled with index 1. Note that for other ECG WMSs in the SS-LUT that use fewer leads, the feature indices may be different. For example, feature indexing for a 3-lead ECG WMS group results in 81 features with index 1 and 198 features with index 3.

The decision maker generation procedure is implemented using WEKA 3-7-13 according to an embodiment of the present invention. Due to the binary classification performed in Tier-1, the labels in the available dataset for DM1 are re-mapped to binary indicators of arrhythmia existence or non-existence. The available datasets for Tier-2 and Tier-3 maintain their 16-class labels. The DM1 generation procedure is explained in detail. DM2 and DM3 are generated in parallel using the same methodology.

In Stage 1, eight base learners are generated from the original available dataset of learners and another eight base learners from the feature-filtered available dataset. These 16 base learners form a base learner candidate pool for the selection of the final decision maker. The base learners include Naive Bayes, Bayes network, SVM, k-nearest neighbor, best-first decision tree, J48, decision table, and multilayer perceptron. Feature filtering is based on supervised forward feature selection. Since feature filtering does not guarantee performance improvement, these 16 base learners and their performance matrices are passed to Stage 2 for meta learner generation and Stage 3 for DM1 generation. Unless otherwise stated, 6-fold cross-validation is used to generate performance matrices. In a 6-fold cross-validation, the original dataset is randomly partitioned into six subsets. In each evaluation round, a new MLM is trained using five subsets and evaluated on the remaining subset. Thus, six models are generated in six rounds. The average performance across the six generated models represents the final performance of this type of MLM.

In Stage 2, six ensemble methods are used to generate the meta learners. These methods include but are not limited to AdaBoost, bagger, voter, stacker, random forest, and random tree. Feature bagging is applied to all 16 base learners. Two types of voters are used: rule based and diversification based. The rule based voter combines base learners with six voting rules: average of probabilities, product of probabilities, majority voting, maximum probabilities, minimum probabilities, and median probabilities. This voter covers max, min, median, and majority rules for posterior probability calculations that generate a final prediction based on base learner classification results. Two separate learner pools are generated for this type of voter, with the first learner pool containing eight base learners from the original set and the other containing eight base learners from the feature-filtered set. The second type of voter, DECORATE, introduces diversification in the learner pool by adding a new voter that disagrees with previous voters on an artificial sample pool. This sample pool contains training instances that are sampled from the original dataset with new labels generated in an inversely proportional fashion to existing predictions. In the current setting, the diversity depth (the number of diversified voters generated) is set to 15, as this was found to be the point of diminishing returns. For stacker, two stacking models are generated using the same learner pools as rule-based voters. Random forest and random tree are implemented separately on these learner pools as well. They are considered homogeneous because they are independent of external base learners. All the generated meta learners from Stage 2 form a meta learner candidate pool for final decision maker generation.

In Stage 3, the final DM1 is obtained using a statistical selector that operates on the base learner and meta learner candidate pools based on a pre-defined selection criterion. Classification accuracy is used for this purpose due to its general effectiveness and widespread use. However, other statistical criterion mentioned previously may be implemented in alternative embodiments.

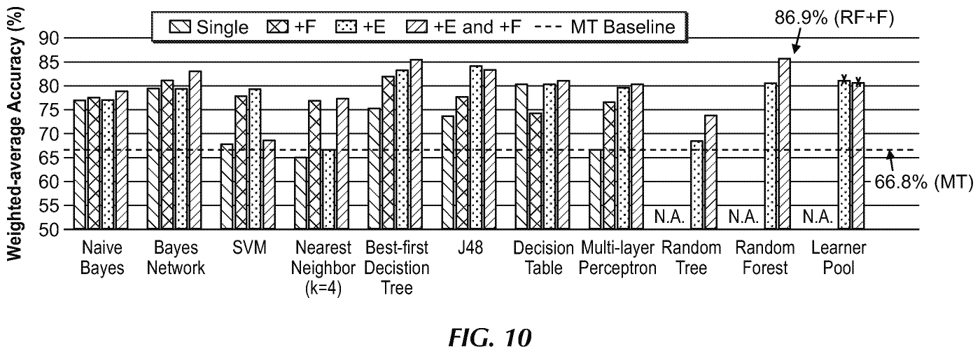

FIG. 10 shows the classification accuracy for the different methods. ‘Single’ represents a base learner that does not use feature filtering or an ensemble method, +F (+E) represents feature filtering (ensemble method), and +E+F represents both. Accuracy can be enhanced through both feature filtering and ensemble methods in most cases. Sometimes, the combination of the two methods gives the best accuracy. From this pool, the best accuracy (85.9%) is obtained for random forest with feature filtering (RF+F). Hence, it is selected as the DM1 for this embodiment.

To compare the machine learning approach disclosed herein against conventional multi-threshold approaches in the WMS-tier, a best-first decision tree model is trained on each of the 279 Tier-1 features individually. This approach splits each possible feature axis into value ranges separated by thresholds and checks the rule's accuracy. In other words, it tries to find the highest accuracy the multi-threshold approach can realize by exhaustively constructing threshold models. The highest accuracy for the conventional multi-threshold approaches was found to be only 69.8%. Thus, the multi-threshold approach is not very accurate.

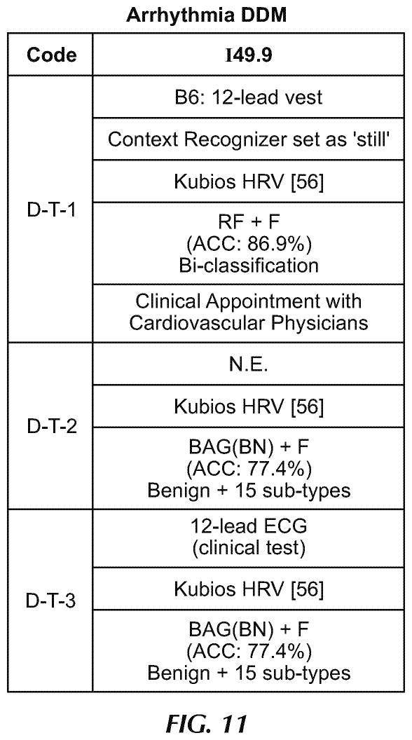

FIG. 11 shows the arrhythmia DDM. The same DDM generation procedure is used to obtain DM2 (ACC: 77.4%) and DM3 (ACC: 77.4%). Note that the drop in their accuracies compared to DM1 arises from their tackling of a more challenging 16-class classification task. Kubios HRV Software is used to extract all 279 features from ECG signals. In the WMS tier, the sensor set code is B6, which matches the 12-lead ECG vest in SS-LUT. The test set codes in Tier-2 and Tier-3 are set to null entry and 12-lead ECG test code, respectively, since clinical 12-lead ECG measurement is a Tier-3 test that requires technician assistance. To get rid of muscle noise, the context recognizer is set to ‘still’, which is within the monitoring capabilities of current WMSs. Finally, the DDM is stamped with its ICD-10-CM code (149.9), and uploaded to the HDSS framework.

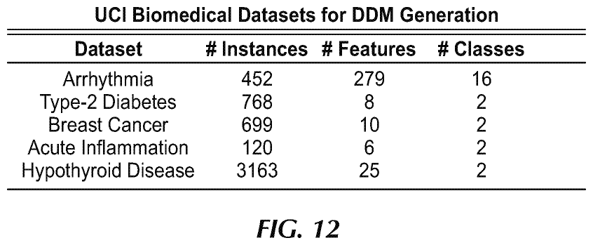

In addition to arrhythmia, DDMs for type-2 diabetes, breast cancer, urinary bladder disorder, renal pelvis origin nephritis, and hypothyroid disease were obtained using publicly available UCI datasets. DDMs for urinary bladder disorder and renal pelvis origin nephritis were generated from the same acute inflammation dataset. FIG. 12 shows the number of data instances, features, and classes in these datasets.

In the feature indexing process for these datasets, Tier-2 tests include triceps skin status checking for type-2 diabetes, and physician lesion observations for urinary bladder disorder, renal pelvis origin nephritis, and hypothyroid disease. Tier-3 tests include oral insulin reaction test for type-2 diabetes, cancer cell microscope test for breast cancer, and blood thyroxine test for hypothyroid disease. All other features acquirable without Tier-2 and Tier-3 tests are indexed with 1, including demographic information, historic disease records, body feelings, ECG, and body temperature measurements. Note that basic body feelings, such as existence of lumbar pain or consistent feelings of pushing urine, are considered Tier-1 features. These features can be transferred to a mobile device by the user through a simple user interface. Thus, they are assumed to be available to the PHDS.

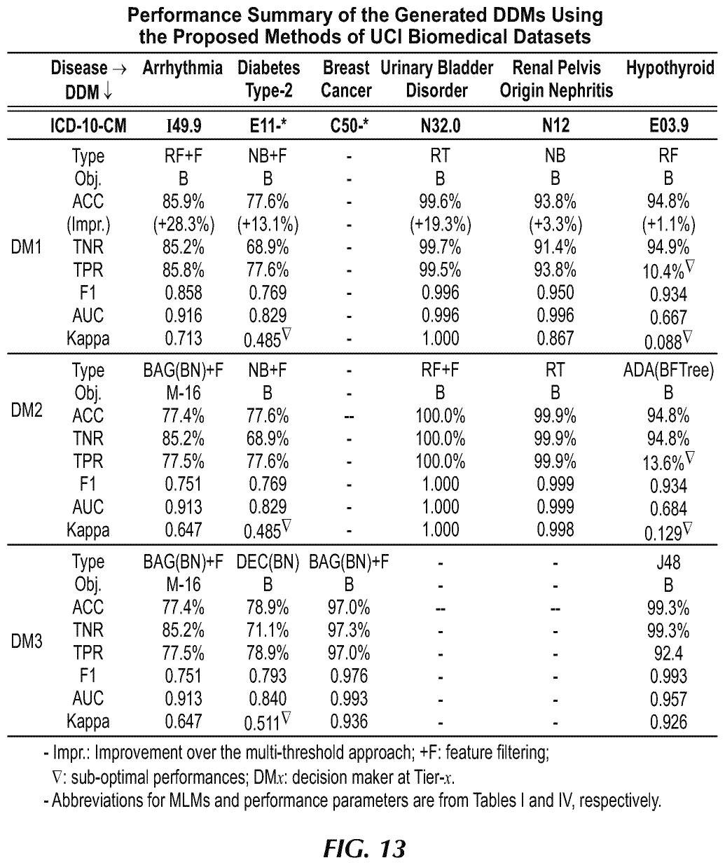

The performance results for the generated DDMs are shown in FIG. 13. In this table, a vertical column represents a DDM for the disease specified at the top. The selected decision maker is shown in the rows indexed with Type. Performance objectives, shown in rows indexed with Obj., vary from binary classifications (B) at Tier-1 to multi-class classification of k classes (M-k) at Tier-2 and Tier-3. The other rows represent corresponding performance metrics, which are stored in performance matrices. A value close to 100% or 1 is ideal for all these measurements. Sub-optimal performances, where future improvements can be made, are highlighted in the table. The percentages shown below DM1 ACCs, indexed as Impr., show the percentage improvement of the machine learning approach relative to the conventional multi-threshold approach.

The quality of training tables, inherited from their original biomedical datasets, plays a significant role in HDSS. A training table with high feature indices may fail to generate decision makers for lower tiers, where the available feature may be null. This limitation arises from the fact that biomedical datasets are often not comprehensive. For example, the breast cancer dataset only contains cell-level features that are only available in Tier-3. Thus, DM3 can only be generated for breast cancer, not DM1 and DM2. If a more comprehensive dataset were available, the DDM generation procedure could generate all three decision makers, as demonstrated in the cases of arrhythmia, type-2 diabetes, and hypothyroid disease. Hence, to fully utilize the power of HDSS, biomedical datasets need to be prepared carefully, updated consistently, and maintained systematically.

Diagnosis for some diseases needs Tier-3 laboratory measurements. Urinary bladder disorder and renal pelvis origin nephritis can be accurately diagnosed within the first two tiers, whereas diseases like hypothyroid rely heavily on clinical laboratory tests. For hypothyroid disease, DM1 and DM2 have very low TPRs. However, these rates increase dramatically with the inclusion of the Tier-3 blood thyroxine test, indicating the importance of this clinical test in its diagnostic process.

Given the need to store a DDM for each of the 69,000 known diseases, an important consideration for establishing the feasibility of HDSS is its tier-wise storage requirement, especially for Tier-1 PHDS that is based on WMSs. In this tier, PHDS may be stored in a cloud server, personal computer base station, or ultimately smartphones for end-user convenience. In all these platforms, use of a moderate amount of storage space would be preferred.

To estimate the storage requirement of the HDSS over all diseases, various base learners are first generated under the WEKA-3-7-13 experimental environment for the arrhythmia dataset, which contains 279 features and 400 training instances. With fewer features, base learners require less storage space. For making a conservative estimate, a feature count of 279 is assumed, which is the worst case among the datasets and the average feature count for the 69,000 diseases. Then, the changes required in storage size are studied when varying the training table sizes and ensemble methods, and using the feature-filtering technique.

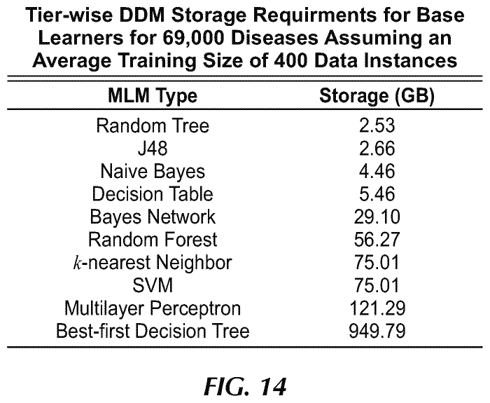

HDSS storage analysis is started with 10 common base learners. For each base learner type, the cumulative storage requirement for 69,000 diseases is given in the corresponding row in FIG. 14. Random tree and random forest are included in this analysis since they are independent of an external base learner. The storage requirement can be seen to vary greatly across different MLM types. Random tree, J48, Naïve Bayes, and decision table require far less storage than Bayes network, random forest, k-nearest neighbor (k=4), SVM, multi-layer perceptron, and best-first decision tree. Their storage requirements fall in a moderate range except for best-first decision tree, which needs nearly 1 TB of storage.

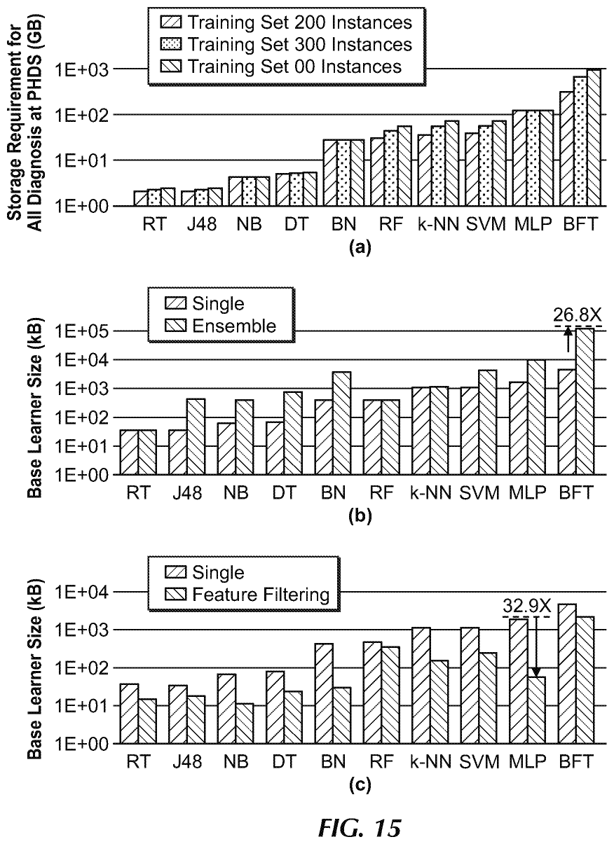

An increase in the number of training instances may incur some storage overheads, as shown in FIG. 15(a). It can be seen that random forest, k-nearest neighbor, SVM, and best-first decision tree are more sensitive to increasing training sizes than the others.

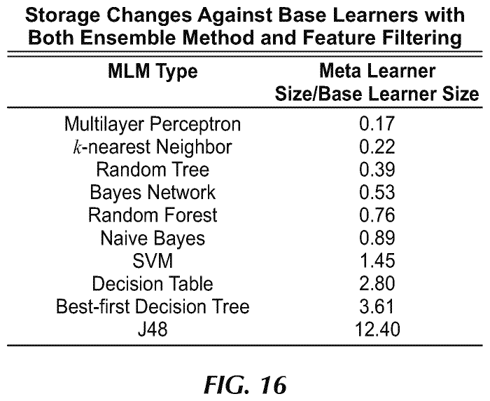

Next, storage requirements under performance enhancement techniques, such as ensemble methods and feature filtering, are studied. As shown in FIG. 15(b), ensemble methods may incur high storage overheads, by as much as 26.8×. This overhead arises from the need to store multiple base learners for a single meta learner. On the other hand, feature filtering enhances the performance at a reduced storage cost, as shown in FIG. 15(c). The storage reduction is as much as 32.9× for multi-layer perceptron. When both enhancement techniques are used simultaneously, the ratio of the meta learner size and base learner size is shown in FIG. 16. A value smaller (greater) than 1 represents a storage reduction (increase). Except for J48 which needs 12.4× the original storage, these changes are generally moderate due to the counteracting pressure from ensemble methods and feature filtering. Therefore, single base learners are used for storage estimation of HDSS.

The tier-wise HDSS storage requirement over all 69,000 diseases is estimated by weighting the base learner storage requirements based on what fraction of time that base learner is used in a 10-year survey of data mining models in the healthcare domain. This yields the following weights: random forest: 44.8%, multilayer perceptron: 17.2%, SVM: 17.2%, decision table: 7.0%, Naive Bayes: 6.9%, Bayes network: 6.9%, and all other base learners weighted zero. Assuming an average training size of 400 instances, the HDSS storage requirement over all 69,000 diseases was estimated to be 61.75 GB (using the storage values for the base learners shown in FIG. 14). Such a storage requirement is completely acceptable in today's cloud server or base station oriented BANs.

As such, disclosed herein is an HDSS that includes WMSs and CDSSs. The HDSS incorporates a hierarchical multi-tier structure supported by robust machine learning. A procedure to generate DDMs is disclosed that can monitor various diseases in parallel. The feasibility of HDSS is demonstrated by generating six DDMs for diseases drawn from four ICD-10-CM categories. It was shown that significant disease classification accuracy can be obtained through physiological data obtained from WMSs themselves. Furthermore, it was discussed how HDSS can also be applied to other disease categories when datasets for those diseases are available. It was estimated that the DDMs for all reported human diseases need around 61.75 GB of storage in the WMSs tier, which is well within the storage means of current technology.

It is understood that the above-described embodiments are only illustrative of the application of the principles of the present invention. The present invention may be embodied in other specific forms without departing from its spirit or essential characteristics. All changes that come within the meaning and range of equivalency of the claims are to be embraced within their scope. Thus, while the present invention has been fully described above with particularity and detail in connection with what is presently deemed to be the most practical and preferred embodiment of the invention, it will be apparent to those of ordinary skill in the art that numerous modifications may be made without departing from the principles and concepts of the invention as set forth in the claims.

1. A hierarchical health decision support system (HDSS) configured to monitor physiological signals, diagnose one or more diseases, and suggest treatment based on data received from one or more wearable medical sensors (WMSs), the system comprising:
a clinical decision support system comprising a diagnosis engine configured to utilize one or more disease diagnosis modules to generate diagnostic suggestions based on the data received from the WMSs, the HDSS being configured with a plurality of tiers to sequentially model general healthcare, the plurality of tiers comprising:
a health decision support tier configured to utilize one or more machine learning models to detect or track one or more diseases based on physiological signals received from the WMSs;
a pre-laboratory decision support tier configured to utilize one or more machine learning models to diagnose one or more diseases based on the received physiological signals and background information; and
a post-decision support tier configured to generate one or more treatment suggestions based on the diagnosed diseases.

2. The HDSS of claim 1 wherein the diagnosis engine comprises a pre-processor, a machine learning engine, and a machine learning database.

3. The HDSS of claim 2 wherein the machine learning engine is configured to utilize one or more of a similarity-based machine learning system, a probabilistic machine learning system, an error-based machine learning system, and an ensemble machine learning system.

4. The HDSS of claim 2 wherein the machine learning engine is configured to utilize one or more of Naïve Bayes, Bayes network, k-nearest neighbor, best-first decision tree, J48 decision table, support vector machine (SVM), and multilayer preceptron.

5. The HDSS of claim 2 wherein the machine learning engine is configured to utilize one or more of Stacker, AdaBoost, Decorate, Bagger, Random tree, and Random forest.

6. The HDSS of claim 1 wherein the plurality of tiers further comprises a post-laboratory decision support tier configured to utilize one or more machine learning models to diagnose one or more diseases based on the received physiological signals and laboratory information.

7. The HDSS of claim 6 wherein the laboratory information comprises data received from one or more laboratory tests performed based on a specific disease candidate.

8. The HDSS of claim 1 wherein the background information comprises data received from clinical questions, clinical observations, and previous health records.

9. The HDSS of claim 1 wherein the treatment suggestions comprise one or more of prescription, medication, and lifestyle suggestions.

10. The HDSS of claim 1 wherein physiological signals comprises one or more of heart rate, body temperature, respiration rate, blood pressure, electroencephalogram, electrocardiogram, Galvanic skin response, oxygen saturation, blood glucose, and body mass index.

11. A method for general healthcare based on a hierarchical health decision support system (HDSS) configured to monitor physiological signals, diagnose one or more diseases, and suggest treatment based on data received from one or more wearable medical sensors (WMSs), the method comprising:
utilizing one or more disease diagnosis modules to generate diagnostic suggestions based on the data received from the WMSs, the diagnostic suggestions being generated by a plurality of tiers comprising:
a health decision support tier configured to utilize one or more machine learning models to detect or track one or more diseases based on physiological signals received from the WMSs;
a pre-laboratory decision support tier configured to utilize one or more machine learning models to diagnose one or more diseases based on the received physiological signals and background information; and
a post-decision support tier configured to generate one or more treatment suggestions based on the diagnosed diseases.

12. The method of claim 11, wherein the health decision support tier further comprises:
selection of target physiological signals;
matching of the target physiological signals with their WMSs;
pre-processing of the target physiological signals for the machine learning models;
diagnostic decision-making through the machine learning models;
obtaining disease signatures of target diseases; and
responding according to the decisions.

13. The method of claim 11, wherein the pre-laboratory decision support tier further comprises utilizing electronic health records and disease onset records to determine the background information.

14. The method of claim 11, wherein the plurality of tiers further comprises a post-laboratory decision support tier configured to utilize one or more machine learning models to diagnose one or more diseases based on the received physiological signals and laboratory information, the laboratory information comprising data received from one or more laboratory tests performed based on a specific disease candidate.

15. The method of claim 11, wherein the post-decision support tier further comprises providing one or more of prescription, medication, and lifestyle suggestions.

16. A method for generating a disease diagnosis module to be utilized by a clinical decision support system, the method comprising:
constructing a training table from a biomedical dataset for a disease;
generating one or more decision-maker modules in a parallel fashion; and
finalizing the disease diagnosis module.

17. The method of claim 16, wherein constructing the training table further comprises a feature indexing process.

18. The method of claim 16, wherein generating the one or more decision-maker modules further comprises:
generating a set of base learners and their performance parameters via one or more performance matrices;
generating a set of meta learners using the base learners; and
comparing the performance matrices of the generated base learner and meta learners via a statistical selector based on pre-defined criteria to select a learner as the decision-maker module.

19. The method of claim 18, where the performance parameters comprise accuracy, true-positive rate, true-negative rate, F1 score, and area under the curve.

20. The method of claim 16, wherein finalizing the disease diagnosis module further comprises packaging the decision-maker module with its performance matrix and learning statistics.
